(19) United States
(12) Patent Application Publication
(10) Pub. No.: US 2025/0288197 A1
(43) Pub. Date: Sep. 18, 2025

(54) DATA RECORDING DEVICE

(71) Applicant: DÜRR DENTAL SE, Bietigheim-Bissingen (DE)

(72) Inventor: Wolf BLECHER, Hemsbach (DE)

(73) Assignee: DÜRR DENTAL SE, Bietigheim-Bissingen (DE)

(21) Appl. No.: 19/073,135

(22) Filed: Mar. 7, 2025

(30) Foreign Application Priority Data
Mar. 12, 2024 (DE) 102024106996.1

(51) Int. Cl. A61B 1/24; A61B 1/00; A61B 1/32

(52) U.S. Cl. CPC A61B 1/24; A61B 1/00154; A61B 1/32

(57) ABSTRACT

The invention relates to a device for data acquisition. The device includes an arrangement, wherein the arrangement can be positioned or received such that at least one end section of the arrangement projects into a cavity, and wherein at least one section of the arrangement is subjected to pressure when the arrangement is positioned such that at least the end section of the arrangement projects into the cavity; and at least one acquisition unit, which is configured and arranged on the arrangement to perform at least one data acquisition of at least a portion of the cavity.

### CROSS-REFERENCE TO RELATED APPLICATIONS

This application claims priority under 35 U.S.C. 119 from German Patent Application No. 102024106996.1, filed Mar. 12, 2024; the disclosure of which is incorporated herein by reference in its entirety.

### FIELD

The present invention generally relates to a device for data acquisition. In particular, the invention relates to a bite block device, a medical imaging system equipped with such a bite block device, and a method for performing an X-ray capture of a region to be imaged in a patient's oral cavity.

### BACKGROUND

Devices and methods for data acquisition are of significance in numerous fields of application. By way of example only (and without limitation), such fields include vehicle diagnostics, wastewater technology, explosives inspection, police operations, and the medical sector. In turn, in the medical field, devices and methods for data acquisition can be employed, for instance, in invasive medical procedures, in the ENT (ear, nose, and throat) domain, and in dentistry.

In medical imaging systems for dental applications, patient positioning is of particular importance. Furthermore, in such systems, it is desirable to select the smallest possible volume for image capture in order, among other things, to reduce the patient's exposure to radiation.

From the prior art, methods are known that use one or two cameras or light localizers to aid an operator in positioning the patient in a digital volume tomography (DVT) unit. All these methods reference patient features that are also visible to the operator. In addition, attempts are made to overlay additional lines on the patient's facial skin or to color-code a tooth chart on the control panel in order to give the operator a sense of which dental region will appear in the DVT scan to be acquired. Because this positioning is not reliably reproducible, the operator typically elects to depict a larger volume in uncertain cases, thereby exposing the patient to an additional radiation dose.

DE 10 2018 212 389 A1 relates to determining an anatomical position based on image data captured with an optical camera of an X-ray device. The optical camera may be arranged on an acquisition assembly (and thus moveable therewith) or be positioned in a fixed location in the X-ray device. The image data generated by the camera show the patient or part of the patient. The image data can also show the position of the acquisition assembly and may, in particular, be real-time image data.

EP 4 140 412 A1 addresses methods for digital volume tomography imaging in the dental field. According to one method, the patient is first positioned in the X-ray device, after which a 3D scout image is generated with a reduced dose, using one of the predefined volume sizes and a predefined rotation center. In a subsequent step, the practitioner marks, on the 3D scout image, the tooth for which high-resolution information is desired, using an enclosing geometry. According to the method sketched, the problem of reliably selecting a minimal volume is addressed by specifically choosing a tooth and calculating how it is projected onto the sensor given a known orbital path. However, this method presupposes a scout acquisition, which already deposits additional dose in the patient, thus nullifying or at least reducing the dose savings achieved by the smaller volume.

In general, there is a need for an improved approach to data acquisition of at least part of a cavity. In particular, there is a need for an improved approach to reliable patient positioning and/or improved selection of a small volume for a dental volumetric scan, specifically a dental DVT.

### SUMMARY

According to a first aspect of the invention, a device is proposed. The device comprises an arrangement and at least one acquisition unit. The arrangement can be positioned or received such that at least one end section of the arrangement projects into a cavity. In other words, the arrangement may be configured to be positioned or received so that at least one end section extends into a cavity. At least a portion of the arrangement is subjected to pressure when the arrangement is positioned or received in such a way that at least the end section of the arrangement projects into the cavity. The at least one acquisition unit is configured and disposed on the arrangement to perform at least one data acquisition of at least a portion of the cavity.

Stated differently, the device, in particular the arrangement, may be designed to have at least one end section that is formed or extends in such a manner that it can project into a cavity when at least a portion of the arrangement is pressurized. The pressurization may arise, in particular, from such positioning or reception of the arrangement that at least the end section of the arrangement projects into the cavity. The device, specifically the arrangement, can be received on or in various objects. If the device, and in particular the arrangement, is positioned on or in an object, then the acquisition unit can carry out at least one data capture of at least a portion of a cavity of that object.

The device can be used for data acquisition in a variety of areas. By way of example only (and without limitation), these areas include vehicle diagnostics, wastewater technology, explosives inspection, police operations, and the medical field. In the medical field, devices for data acquisition can be used, for example, in invasive medical procedures, in the ear, nose, and throat (ENT) domain, and in dentistry.

The device may, for example, include or be configured as a bite block device or another suitable holding structure. Alternatively, the device may include or be formed as an endoscope, a catheter, or another medical device that can be introduced into a body opening. The device could also include or be configured as a non-medical endoscope.

The cavity may be or include a body opening, such as an oral cavity, one or more arteries, or cardiac ventricles. Alternatively, the cavity could be or include a room (e.g., a living space), a boiler, a tank, a silo, or a cavity in buildings or machinery (e.g., a submarine pipe system). The data acquisition may include or be configured as the capture of image information, photons, or pixels. The pressurization may be accomplished by receiving the arrangement in an object. For example, the pressurization may result from placing the device-specifically, the arrangement-between teeth, on suction knobs, on adjustable endoscope mounts, on or beneath a door, or on other mounting components.

The at least one acquisition unit can be formed on the end section of the arrangement. In this way, the acquisition unit can easily perform data capture when at least the end section projects into the cavity.

For instance, the end section of the arrangement may be configured such that it is not pressurized when the arrangement is positioned or received so that at least the end section of the arrangement projects into the cavity. Hence, although at least one section (e.g., the section placed in the object) is pressurized, the end section is not. This allows the end section to project reliably into the cavity. This is particularly advantageous when the acquisition unit is disposed on the end section.

Additionally, the device can have at least one emitting unit. The emitting unit can be arranged on the arrangement. The emitting unit may include, or be formed as, a light source, an X-ray source, a laser source, an infrared source, an ultraviolet (UV) source, a terahertz-wave source, and/or a high-frequency (HF) pulse source.

The at least one emitting unit may be formed on the end section of the arrangement. For example, both the acquisition unit and the emitting unit may be formed on the end section. In this way, the acquisition unit and the emitting unit can jointly illuminate and/or irradiate at least a portion of the cavity when at least the end section projects into the cavity.

The emitting unit can be configured and arranged on the arrangement such that, in cooperation with the acquisition unit, it images at least part of the cavity. For example, data capture of at least part of the cavity can be performed through cooperation between the acquisition unit and the emitting unit.

The emitting unit can include or be formed as at least one illumination component. This illumination component can be configured and arranged on the arrangement so as to illuminate at least part of a patient's oral cavity. The at least one illumination component may comprise a light source, e.g., an LED, or be formed as such an LED.

The device can be configured to forward the at least one data capture to a higher-level system for further use. The higher-level system can employ the data acquisition to influence an independent system. The independent system may be independent of the device and/or of the higher-level system. The independent system can include at least a portion of a human body or a medical imaging system. For instance, the data acquisition might be used to position at least part of the human body or to obtain information about at least a portion of the human body, such as a tumor.

According to a second aspect, a bite block device for a medical imaging system, particularly a dental X-ray system, is proposed. The bite block device comprises a bite arrangement and at least one camera. The bite arrangement is configured to be positioned or placed between at least one tooth of the upper jaw and at least one tooth of the lower jaw of a patient so that at least an end section of the bite arrangement projects into the patient's oral cavity. In other words, the bite arrangement is designed to be positioned between at least one tooth of the upper jaw and at least one tooth of the lower jaw of a patient in such a way that at least one end section of the bite arrangement extends into the patient's oral cavity. The at least one camera is configured and arranged on the bite arrangement so as to capture/create/produce at least one image of at least part of the patient's oral cavity.

The at least one image may be referred to as at least one optical image. The at least one camera may be referred to as at least one optical camera. The at least one portion of the oral cavity may include one or more teeth or be constituted by one or more teeth.

The at least one camera can be formed on the end section of the bite arrangement. During placement of the bite arrangement between the patient's teeth, the end section can project into the patient's oral cavity. For example, the end section could be cylindrical. The end section can have a circular or rectangular cross section. A flat end face of the cylindrical end section, for instance, can incorporate the at least one camera. Depending on perspective, the end section can be T-shaped, U-shaped, or V-shaped. Both flat end faces of a T-shaped, U-shaped, or V-shaped end section, for example, can each incorporate one of the at least one camera. Additionally, or alternatively, one or more cameras of the at least one camera can be arranged on an arc or arm of a U-shaped or V-shaped end section.

Furthermore, the bite block device may include at least one illumination component. The at least one illumination component can be arranged and configured on the bite arrangement to illuminate at least a portion of the patient's oral cavity. The portion of the patient's oral cavity illuminated by the at least one illumination component can correspond at least approximately to the portion of the oral cavity recorded by the at least one camera.

The at least one illumination component can be formed on the end section of the bite arrangement. The end section could be cylindrical, for example, with a circular or rectangular cross section. A flat end face of a cylindrical end section can incorporate the at least one illumination component. The at least one camera and the at least one illumination component can be arranged together on the same end face. The at least one illumination component can surround the at least one camera or vice versa. Alternatively, the end section can be T-shaped or U-shaped. Both flat end faces of a T-shaped or U-shaped end section can each include a camera from the at least one camera, and the at least one illumination component can be placed separately from the camera on another part of the bite arrangement. The at least one illumination component can be or include one or more LEDs.

The at least one illumination component can be placed on the bite block device, in particular on the end section of the bite block device, separately from the camera(s). Alternatively, the at least one illumination component can be arranged together with the camera(s) on the bite block device, in particular on the end section. For example, as an LED or an LED array, the illumination component can encircle the at least one camera in a ring, or vice versa.

According to a third aspect, a medical imaging system is proposed. The medical imaging system can comprise a bite block device according to the second aspect and an X-ray recording device. The bite block device according to the second aspect comprises a bite arrangement and at least one camera. The bite arrangement is configured to be placed or received between at least one tooth of the upper jaw and at least one tooth of the lower jaw of a patient so that at least one end section of the bite arrangement projects into the patient's oral cavity. Put differently, the bite arrangement is designed to be positioned between at least one tooth of the upper jaw and at least one tooth of the lower jaw of a patient so that at least one end section of the bite arrangement extends into the patient's oral cavity. The at least one camera is configured and arranged on the bite arrangement so as to capture/create/produce at least one image/optical image of at least part of the patient's oral cavity. The X-ray recording device is configured to perform at least one X-ray capture of a region of the patient's oral cavity to be imaged, considering information about the image of at least part of the patient's oral cavity. In other words, when performing/acquiring an X-ray image of the region to be imaged in the patient's oral cavity, the information regarding the image of at least part of the patient's oral cavity can be used or considered. For example, the at least one X-ray capture of the region to be imaged in the patient's oral cavity can be performed in consideration of the image of at least part of the oral cavity.

The medical imaging system can include or be configured as a dental X-ray device. In particular, the medical imaging system can be or include an extraoral dental X-ray device.

The region of the X-ray capture to be imaged can correspond at least approximately to that portion of the oral cavity. Alternatively, the region of the X-ray capture can differ at least in part from that portion of the oral cavity.

The at least one X-ray capture of the region to be imaged can be or include at least one high-resolution and/or three-dimensional X-ray scan. The at least one X-ray capture of the region to be imaged can be or include at least one digital volume tomography (DVT) scan. DVT customarily denotes a three-dimensional imaging tomography procedure using X-rays. Similar to CT (computed tomography) or MRI (magnetic resonance imaging), DVT also generates sectional images. DVT devices create their volumetric datasets by means of a mathematical process (back projection) from typically several hundred individual X-ray projection images. Unlike two-dimensional X-ray images, in which information in the direction of the beam path is significantly reduced, three-dimensional X-ray (e.g., DVT) allows depiction of the imaged anatomical structures in all spatial directions.

The medical imaging system may also include a computing unit. The computing unit may be configured to determine the region of the patient's oral cavity to be imaged, taking into account the information about the image of at least part of the patient's oral cavity. The computing unit can be configured to do so automatically. Alternatively, this region of the oral cavity to be imaged may be input manually by an operator, using the image of at least part of the oral cavity. For instance, the operator may select the region to be imaged on the image of that portion of the oral cavity.

Additionally, or alternatively, the computing unit may be configured to determine information about a trajectory curve of the medical imaging system based on the information about the image of at least part of the patient's oral cavity. The computing unit can, for example, determine information about the trajectory curve based on that image. Such trajectory information might include one or more rotation centers of the imaging system's orbit. It may also include information about orbit speed or details of the orbital path. It could comprise data on a rotation speed of a rotating radiation source (e.g., an X-ray source) and/or a detector, in particular an X-ray detector.

As noted above, the medical imaging system can include or be an extraoral X-ray device. The extraoral X-ray device can be configured as a panoramic X-ray unit, a DVT device, or a hybrid system combining both a panoramic and a DVT device. The X-ray device may include an X-ray emitter and an X-ray detector. The emitter and/or detector may travel along a predefined or determinable trajectory curve, especially around a portion of a patient's head. A standard trajectory curve may be stored or saved in the imaging system and executed without reference to patient-specific details. Based on the information obtained from at least one captured image, the standard trajectory can be adapted. In particular, a rotation center or multiple rotation centers of the standard trajectory can be adjusted. Additionally, a collimator position and/or an image-capture position on a detector (especially an X-ray detector) in the medical imaging system can be adapted.

The bite block device (e.g., the bite arrangement and/or the at least one camera) can be connected or connectable to the computing unit wirelessly or via cable, i.e., the link between the bite block device (e.g., the bite arrangement and/or the at least one camera) and the computing unit can be via a wired connection, a wireless connection, or a combination of both. Through that connection, the bite block device (e.g., the bite arrangement and/or the at least one camera) can transmit the at least one image it has captured to the computing unit.

The medical imaging system can include an output unit. The output unit can be configured to output the image of at least part of the oral cavity. Specifically, the output unit can be configured to display an overlay of the image of at least part of the oral cavity with the region of the patient's oral cavity to be imaged.

The medical imaging system can also include an input unit. The input unit can be combined with or formed on the output unit, for instance. For example, the input unit might include one or more buttons and/or touchscreen elements on the output unit. The input unit can be configured to receive an input or selection of a predefined volume size for the region to be imaged. Additionally, or alternatively, the input unit can be configured to receive a mark or selection of the region to be imaged on the image of at least part of the patient's oral cavity.

The at least one camera can be configured to capture at least one intermediate image of at least part of the patient's oral cavity during the acquisition of the X-ray of the region to be imaged in the patient's oral cavity. This intermediate image can be used to provide information about or detect potential patient movement. In this way, image artifacts can be reduced or even minimized.

According to a fourth aspect, a method is proposed for performing an X-ray scan of a region to be imaged in a patient's oral cavity. The method includes:
positioning or placing (such that it is received) a bite arrangement of a medical imaging system between at least one tooth of the upper jaw and at least one tooth of the lower jaw of a patient, so that at least an end section of the bite arrangement extends into the patient's oral cavity;
performing at least one image capture of at least part of the patient's oral cavity using at least one camera arranged on the bite arrangement; and
performing an X-ray scan of at least one region to be imaged in the patient's oral cavity with an X-ray recording device of the medical imaging system, taking into account information about the image of at least part of the patient's oral cavity.

A fifth aspect pertains to a computer program having program code means which, when loaded into a computer or processor (for instance, a microprocessor, microcontroller, or digital signal processor (DSP)), or when run on a computer or processor (e.g., microprocessor, microcontroller, or DSP), causes the computer or processor to carry out one or more steps or all steps of the method steps described above with respect to the computing unit and/or the X-ray acquisition. A program storage medium or computer program product containing the aforementioned computer program is also provided.

For instance, the computer program according to the fifth aspect may be stored in the computing unit of the medical imaging system according to the third aspect and may cause the computing unit to implement one or more or all of the aspects and/or method steps previously described with respect to the computing unit. Similarly, the computer program according to the fifth aspect can be stored in the computing unit of the medical imaging system according to the third aspect and can cause the computing unit to perform one or more or all of the details, aspects, and/or features described above with respect to the computing unit.

Although some details have been described above in relation to the device according to the first aspect or the bite block device according to the second aspect, these features can also be implemented correspondingly in the medical imaging system, the method, or a computer program implementing the method or the computing unit, and vice versa. Likewise, the aspects described above in connection with the method can be realized in the medical imaging system, the device, or a computer program implementing the method or the computing unit, and vice versa.

### BRIEF DESCRIPTION OF THE DRAWINGS

The present disclosure will be further explained on the basis of figures. These figures schematically show: FIG. 1 shows a skull with a conventional bite splint;

FIG. 2 shows a bite block device according to an exemplary embodiment in a side view;

FIG. 3 shows a bite block device according to an exemplary embodiment in a top view;

FIG. 4 shows a medical imaging system with a bite block device according to the exemplary embodiments of FIGS. 2 and 3;

FIG. 5 shows an output unit that can be employed with a bite block device according to the exemplary embodiments of FIGS. 2 and 3 and/or the medical imaging system of FIG. 4;

FIG. 6 shows a flow diagram of a method according to an exemplary embodiment.

### DETAILED DESCRIPTION

Specific details are given below, purely by way of example and without limitation, to facilitate a thorough understanding of the present disclosure. It will be clear to the person skilled in the art, however, that the present disclosure may be practiced in embodiments that differ from the details set out below. For example, in the following, a bite block device is specifically described as an example of a device for data acquisition. Other designs of the device for other uses are likewise conceivable and feasible. In addition, specific configurations and designs of a medical imaging system and/or a bite block device with a bite arrangement are described below, without implying any limitation.

It will be clear to a person skilled in the art that the following explanations may be implemented by means of hardware circuits, software means, or a combination thereof. The software means may be associated with programmed microprocessors, artificial intelligence, or a general-purpose computer, an ASIC (application-specific integrated circuit), and/or DSPs (digital signal processors). It is also evident that even when the details described below relate to a method, these details may be realized in a suitable device unit, a computer processor, or a memory connected to a processor, where the memory holds one or more programs that execute the method when run by the processor.

The exemplary embodiments may be realized and/or employed in various medical imaging systems. By way of example only, these include X-ray devices, in particular panoramic X-ray devices, DVT X-ray devices, or hybrid devices that incorporate both a panoramic and a DVT X-ray device.

FIG. 1 schematically illustrates a patient's skull and a conventional bite splint 1. As shown, the patient bites into the bite splint 1 before and/or during the performance of X-ray scans. Through the engagement of the teeth with the bite splint, a partial fixation of the patient's head is hoped for and especially that the head remain as still and in as fixed or calibrated a position as possible during the X-ray capture(s). In particular, the aim is to reduce or nearly prevent skull translation by having the patient bite down. In practice, however, this approach is error-prone. First, the head often does not remain in the desired still position. Second, the positioning depends on the experience of the clinical staff and thus is subject to human error or misjudgment.

In dental medical imaging systems, patient positioning is of particular importance. Furthermore, minimizing the volume selected for imaging is desirable in such systems, e.g., to reduce radiation exposure. However, because patient positioning and hence the choice of region to be imaged are not reliably reproducible, in uncertain cases the operator typically includes a larger volume, thus administering an additional radiation dose to the patient.

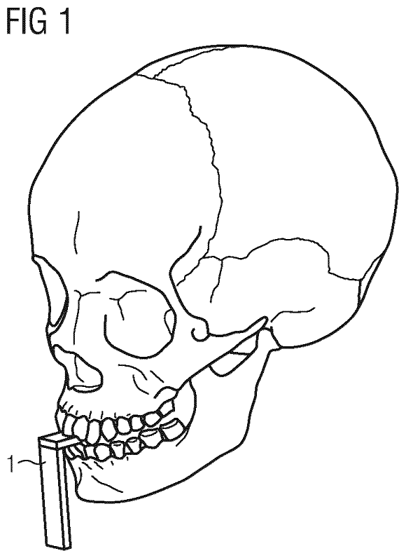

FIG. 2 schematically shows a bite block device 10 in a side view according to one exemplary embodiment. The bite block device 10 is suitable for a medical imaging system, especially a dental X-ray device. The bite block device 10 comprises a bite arrangement 100, which can in particular be configured as or comprise a bite splint. The bite arrangement 100 is configured to be placed or accepted between at least one tooth of the upper jaw and at least one tooth of the lower jaw of a patient so that at least an end section of the bite arrangement 100 projects into the patient's oral cavity. The basic positioning of a bite splint 1 between the patient's teeth is shown in FIG. 1 and was outlined above in connection with that figure.

By way of example, in FIG. 2, the bite block device 10 includes precisely one camera 110 to illustrate that at least one camera can be provided. The camera 110 is arranged on the bite arrangement 100. It is configured and positioned on the bite arrangement 100 so as to capture at least one image/optical image of at least part of the patient's oral cavity.

To accomplish this, the camera 110 is located on or in the end section of the bite arrangement 100 that is introduced between the patient's teeth in proper use and thus extends into the oral cavity. In FIG. 2, the camera 110 is located on an end face of the bite arrangement 100. More precisely, it is situated on an end face (or base face) of the portion of the bite arrangement 100 that is shown by way of example to be partly tubular or cylindrical.

Furthermore, by way of example, the bite block device 10 in FIG. 2 includes exactly one illumination component 120 to illustrate that at least one such component can be provided. The illumination component 120 is arranged on the bite arrangement 100 in such a way that it can illuminate at least part of the patient's oral cavity.

For that purpose, the illumination component 120 is also situated on the end section of the bite arrangement 100 that is inserted between the patient's teeth in proper use and extends into the oral cavity. In FIG. 2, the illumination component 120 is formed on the end face of the partly tubular or cylindrical end section of the bite arrangement 100. In this example, the illumination component 120 is circular or ring-shaped and fully encircles the camera 110 in that ring shape.

In FIG. 2, the camera 110 and the illumination component 120 are arranged together on the same face, i.e., the end face or base face, of the bite arrangement 100. Consequently, the camera 110's viewing angle and the illumination direction of the illumination component 120 coincide at least approximately, so the portion of the oral cavity that the camera 110 aims to capture can be efficiently illuminated by the illumination component 120.

Additionally, the bite block device 10 has a bite recess 130 in the example of FIG. 2. The bite recess 130 is formed in the bite arrangement 100 (e.g., in a bite splint) of the bite block device 10. In FIG. 2, the bite recess 130 is shown only on the top side of the bite arrangement 100 for receiving at least one tooth of a patient's upper jaw. Additionally, or alternatively, a corresponding recess can be formed on the underside of the bite arrangement 100 for receiving at least one tooth of the patient's lower jaw. The bite recess 130 is positioned so that, during proper use of the bite block device 10 between the patient's teeth, the camera 110 and the illumination component 120 point toward and/or are inserted into the patient's oral cavity, allowing at least part of that oral cavity to be captured and illuminated.

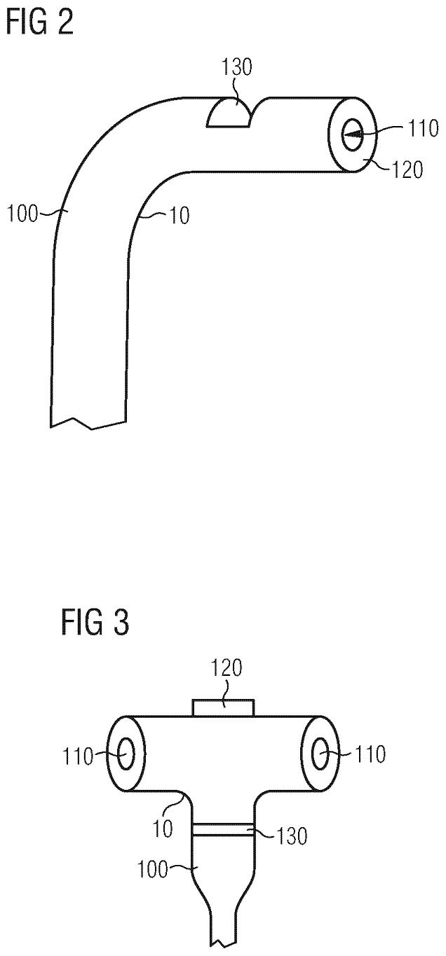

FIG. 3 schematically shows, in top view, another exemplary embodiment of a bite block device 10. The device 10 in FIG. 3 can be seen as a variant of the bite block device 10 in FIG. 2. Accordingly, details described with respect to FIG. 2 will not be repeated at length here.

In FIG. 3, the bite block device 10, by way of example, has two cameras 110 to illustrate that one or more cameras may be present. These cameras 110 are arranged on the bite arrangement 100, configured to capture at least one image/optical image of a portion of the patient's oral cavity.

They are positioned on or in the end section of the bite arrangement 100 that is introduced between the patient's teeth in proper use, thus extending into the oral cavity. In FIG. 3, each of the cameras 110 is positioned at an end face of the bite arrangement 100.

When viewed from above as shown in FIG. 3, the end section of the bite arrangement 100 can be referred to as T-shaped. Here, the cameras 110 are placed respectively on one end face or base face of the T-shaped end section. Additionally, the crossbar of the “T” can be slightly curved upward or downward-when viewed frontally toward the illumination component-to form a U shape or horseshoe shape or parabolic shape. Each end of the “U” can hold one of the cameras 110. In general, the T-shape shown in FIG. 3 is merely an example. Alternatively, or additionally, a U-shape, parabolic shape, or V-shape might be used. The shape can be adapted to the patient's dental arch. The cameras can be placed at each end or at other positions (e.g., along the arc or arms of a U-shape or V-shape) and oriented accordingly.

Additionally, in FIG. 3, there is exactly one illumination component 120 to illustrate that one or more such components may be present. The illumination component 120 is arranged on the bite arrangement 100, spaced from the two cameras 110. In the T-shaped end section, for instance, the illumination component 120 is situated in the center region of the T's crossbar. If the end section is U-shaped or parabolic or horseshoe-shaped, the illumination component 120 can be placed at the apex. At any rate, the illumination component 120 is arranged and configured on the bite arrangement 100 to illuminate at least a portion of the patient's oral cavity.

In the example of FIG. 3, the illumination component 120, as described, is also located within the end section of the bite arrangement 100 that extends into the patient's oral cavity during proper use. Unlike the example in FIG. 2, it is not ring-shaped or arranged around the cameras 110. Instead, it protrudes toward the oral cavity from the T-shaped or U-shaped end section so as to illuminate the space within the mouth.

Hence, although in FIG. 3 the cameras 110 and the illumination component 120 are not on the same face of the bite arrangement 100, it is still ensured that the illumination component 120 lights at least part of the oral cavity and that the cameras 110 face at least some portion of the illuminated area. Thus, the two cameras 110 can still efficiently capture the region of the oral cavity that is illuminated by the illumination component 120.

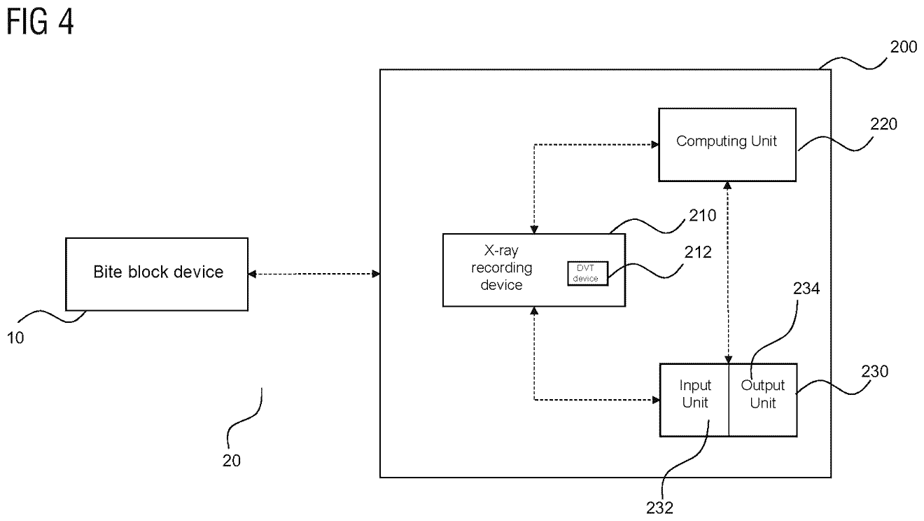

FIG. 4 is a block diagram of a medical imaging system 20, especially a dental X-ray system. The medical imaging system 20 comprises a bite block device 10 which may be configured as described in connection with FIGS. 2 and 3. Thus, the bite block device 10 includes a bite arrangement 100, which may be or include a bite splint. The bite arrangement 100 is configured to be received between at least one tooth of the upper jaw and at least one tooth of the lower jaw of a patient so that at least a portion of the bite arrangement 100 projects into the patient's oral cavity. The bite arrangement 100 has at least one camera 110 that is configured and arranged on the bite arrangement 100 to capture/create/produce at least one image (optical image) of at least part of the patient's oral cavity.

By way of example, the medical imaging system 20 includes an X-ray device 200, for instance a panoramic X-ray device. The medical imaging system 20—and in particular the X-ray device 200—includes an X-ray recording device 210. The X-ray recording device 210 is configured to acquire at least one X-ray image of a region to be imaged in the patient's oral cavity, considering information about the optical image of at least part of the patient's oral cavity. The X-ray recording device 210 may, for example, incorporate a DVT device 212 configured to generate at least one volumetric X-ray scan (in particular, at least one DVT scan) of the region to be imaged.

Moreover, the medical imaging system 20—and specifically the X-ray device 200—can include a computing unit 220, which can be any suitable software and/or hardware such as one or more processors. The computing unit 220 is in communication (wired and/or wireless) with the X-ray recording device 210 to exchange the necessary information. The computing unit 220 is configured to determine the region of the patient's oral cavity to be imaged, taking into account the information about the optical image of at least part of the oral cavity. This determination may occur automatically or partially under operator input.

Additionally, or alternatively, the computing unit 220 may determine information regarding a trajectory curve of the medical imaging system 20 based on the information from the optical image. Such information could include a rotation center or multiple rotation centers of the orbit, or data about the rotation speed, path, or scanning geometry.

The bite block device 10—i.e., the bite arrangement 100 and/or the at least one camera 110—can be connected or connectable (wired or wireless) to the X-ray device 200 and thus to the computing unit 220. Via that connection, the bite block device 10 can transmit the at least one captured image from the camera 110 to the X-ray device 200 and the computing unit 220, in particular before the X-ray scan is performed. Additionally, the camera 110 can acquire one or more intermediate images of at least part of the patient's oral cavity while the X-ray is being taken and send them to the X-ray device 200 and computing unit 220. Using these, the imaging system (particularly the computing unit 220) can detect or derive a possible patient movement and compensate for that in the X-ray scan to reduce motion artifacts.

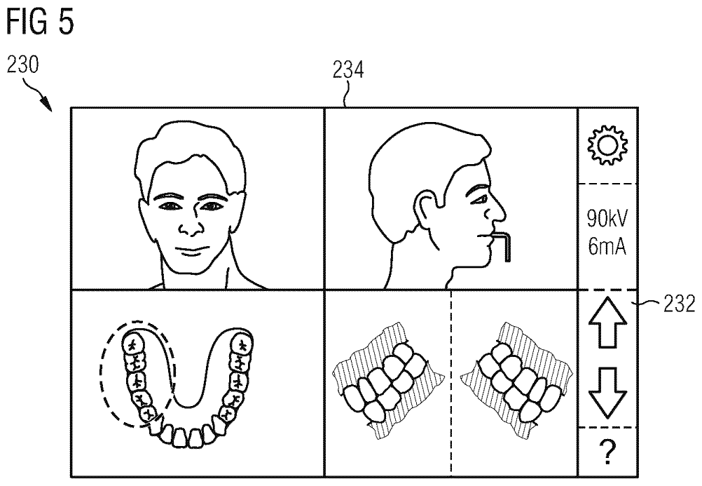

Furthermore, the medical imaging system 20—and specifically the X-ray device 200—may include an input-output devicedevice 230 comprising, for instance, an input unit 232 and an output unit 234. In FIG. 4, for example, these are shown as a shared input-output device 230, although they may also be realized separately. The output unit 234 is configured to display the image of at least part of the oral cavity. In particular, it can display an overlay of the image of the oral cavity with the region of the oral cavity to be imaged. Example details of such an output unit 234 appear in FIG. 5, which is described below.

The input unit 232 is configured to receive an input or selection of a predefined volume size for the region to be imaged. Additionally, or alternatively, it can be configured to receive a marking or selection of the region to be imaged on the displayed image of at least part of the patient's oral cavity. The input unit 232 may incorporate one or more buttons and/or touchscreen elements on the output unit 234, in the case that the input and output are combined into a single interface. The input unit 232 can receive, for example, the input specifying the region to be imaged. The region in the patient's mouth can be selected by a user on the displayed image of the patient's oral cavity.

FIG. 5 provides an example of such a combined input-output device 230 that can be used in the medical imaging system 20 of FIG. 4. On the right side, the input unit 232 has multiple input fields or keys for parameter selection, adjusting X-ray parameters, controlling the vertical positioning of the imaging system, and invoking a help function, among others. These examples can be extended or replaced as needed.

To the left of the input unit 232 is the output unit 234, which has multiple output fields for displaying various information. For instance, one or more of these fields can show camera images from extraoral cameras. Additionally, a field can display images captured by the one or more cameras 110 from FIG. 3. On such images, an operator can select or determine the region to be imaged. That region can then be depicted as a chosen volume on a tooth chart displayed in another field of the output unit 234, where it may be further adjusted if needed.

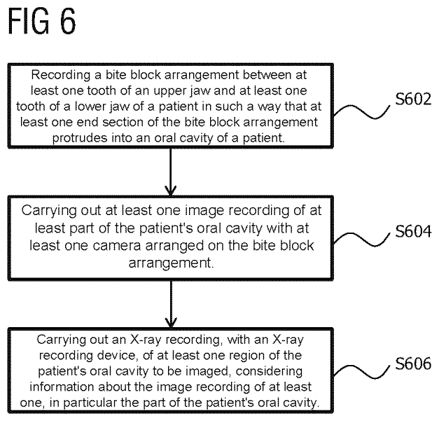

FIG. 6 shows a flow diagram of a method for taking an X-ray image of a region of a patient's oral cavity to be imaged. The method comprises:

(S602) The method comprises recording a bite block arrangement between at least one tooth of an upper jaw and at least one tooth of a lower jaw of a patient in such a way that at least one end section of the bite block arrangement protrudes into an oral cavity of a patient.

(S604) The method comprises carrying out at least one image recording of at least part of the patient's oral cavity with at least one camera arranged on the bite block arrangement.

(S606) The method comprises carrying out an X-ray recording, with an X-ray recording device, of at least one region of the patient's oral cavity to be imaged, considering information about the image recording of at least one, in particular the part of the patient's oral cavity.

Additional details of the devices and methods described with respect to FIGS. 1-6 namely, the bite block device 10 from FIGS. 2 and 3, the medical imaging system 20 from FIG. 4, and the method from FIG. 6 are set out below.

By means of a camera 110 placed on or in a bite block device 10, an image of the mouth interior/oral cavity is displayed during patient positioning. After selecting a predefined volume, the teeth contained in that volume are correspondingly marked on the captured image (e.g., colored). Alternatively, the operator of the medical system 20 can select a series of teeth on the mouth interior/oral cavity image. Using appropriate algorithms, the system calculates the trajectory by which the corresponding corner points will be projected onto the sensor/detector, enabling a volume capture tailored to the region of interest.

Placing one or more cameras 110 plus one or more illumination aids 120 on/in the bite block device 10 permits displaying an image of the oral cavity alongside other positioning aids. When selecting a predefined volume size, based on the known reference position of the bite block device 10, the image of the oral cavity as well as possibly an extraoral camera image can be overlaid with the region to be captured so that the operator of the medical system 20 can see not only the outside of the patient but also directly within the dental region which area will be imaged.

Alternatively, the operator may select the region to be imaged directly on the camera image. With the known reference position of the bite block device, together with, for example, a width measurement via temple supports or other aids, the region to be imaged within a larger, known volume can be established, and the corresponding location on the detector of the medical system 20 can be computed using suitable algorithms. By adjusting the collimator of the medical device 20, only the selected portion of the oral cavity is imaged by the device (as opposed to the cameras) in the DVT scan. Alternatively, a suitable orbit for the rotation center can be chosen that matches the region to be imaged, and the scan is performed accordingly.

Another advantage of the bite camera 110 is that it can generate an optical image of the oral cavity during the X-ray scan. By comparing existing markers (e.g., interdental spaces) or using other known techniques, one can detect potential patient movement during the scan and use that information to correct motion artifacts.

The additional information from the oral cavity enables the operator to reliably position the patient and carry out a DVT and/or panoramic scan adapted to the diagnostic question, thereby minimizing dose for the patient.

### REFERENCE NUMBER LISTING

Bite block device 10
X-ray recording device 210
DVT device 212
Computing unit 220
Input-output device 230
Input unit 232
Output unit 234

1. A device comprising:
an arrangement, wherein the arrangement can be positioned or received such that at least one end section of the arrangement projects into a cavity, and wherein at least one section of the arrangement is subjected to pressure when the arrangement is positioned such that at least the end section of the arrangement projects into the cavity; and
at least one acquisition unit, which is configured and arranged on the arrangement to perform at least one data acquisition of at least a portion of the cavity.

2. The device according to claim 1, wherein the at least one acquisition unit is formed on the end section of the arrangement.

3. The device according to claim 1, wherein at least the end section of the arrangement is not subjected to pressure when the arrangement is positioned such that at least the end section of the arrangement projects into the cavity.

4. The device according to any of claim 1, further comprising:
at least one emitting unit, which is arranged on the arrangement.

5. The device according to claim 4, wherein the at least one emitting unit is formed on the end section of the arrangement.

6. The device according to claim 4, wherein the at least one emitting unit is a light source, an X-ray source, and/or a laser source.

7. The device according to any of claim 4, wherein the at least one emitting unit is configured and arranged on the arrangement in such a way that, in interaction with the acquisition unit, the at least one emitting unit is configured to image at least a portion of the cavity.

8. The device according to any of claim 1, wherein the device is configured to transmit the at least one data acquisition to a higher-level system for further use.
